(19) United States
(12) Patent Application Publication
(10) Pub. No.: US 2024/0172791 A1
(43) Pub. Date: May 30, 2024

(54) Inhalation Device For Delivering An Atomized Mixture Of Liquids With A Regulated Mixing Ratio

(71) Applicant: DP Innovations Kft., Nyiregyhaza (HU)

(72) Inventors: Tibor Ajpli, Batonytereney (HU); Tamas Kolb, Budapest (HU); Balazs Polacesk, Balassagyarmat (HU); David Uzoni, Budapest (HU)

(73) Assignee: DP Innovations Kft., Nyiregyhaza (HU)

(21) Appl. No.: 18/552,358

(22) PCT Filed: Feb. 21, 2022
(86) PCT No.: PCT/HU2022/050012

§ 371 Date: Sep. 25, 2023

(30) Foreign Application Priority Data
Mar. 29, 2021 (HU) P2100130

(51) Int. Cl. A24F 40/30; A24F 40/10; A24F 40/50; A61M 15/00; A61M 16/00

(52) U.S. Cl. CPC A24F 40/30; A24F 40/10; A24F 40/50; A61M 15/0003; A61M 15/0066; A61M 16/0003; A61M 2016/0024; A61M 2016/0027; A61M 2205/3334; A61M 2205/8206

(57) ABSTRACT

The invention is an inhalation device for delivering an atomized mixture of liquids with a regulated mixing ratio, comprising
two reservoirs storing a liquid,
for each reservoir a piezoelectric actuator (10a, 10b) for atomizing the liquid, and a liquid transferring member being in contact with the liquid and the piezoelectric actuator (10a, 10b), and
a pressure sensor (16a, 16b) for detecting a user inhalation,
a regulating electric circuit comprising a controller (18) generating an output PWM signal having a duty cycle, and an output stage (20a, 20b) comprising
   an inductor (12a, 12b) forming a resonant circuit with the piezoelectric actuator (10a, 10b), and
   a switch (14a, 14b) for driving the output stage (20a, 20b) by the output PWM signal,
the mixing ratio of the atomized liquids is determined by the duty cycles of the output PWM signals.

### TECHNICAL FIELD

The invention relates to an inhalation device for delivering an atomized mixture of liquids having a variable mixing ratio. Inhalation devices can be medical devices for delivering one or more active component into the lung of a patient, or electric cigarettes (e-cigarettes) atomizing liquid nicotine and/or other substances into the lung of a user.

### BACKGROUND ART

US 2010/0200008 A1 discloses an e-cigarette, wherein a sponge storing a liquid is in contact with an ultrasonic atomizer.

US 2012/0048266 A1 and US 2017/0135410 A1 disclose inhalation devices and e-cigarettes being capable of atomizing liquids stored in separate reservoirs by a piezo atomizer and mixing the atomized liquids in a predetermined ratio. The documents do not disclose the contact of the liquid and the piezo atomizer and also do not disclose the details of the exact mixing of the liquids. Furthermore, US 2012/0048266 A1 does not disclose implementations of a precise regulation of the inhalation device.

US 2016/0089508 A1 also disclose an inhalation device for vaporizing and mixing two liquids. The evaporation can be implemented by a piezo atomizer; however, the document does not disclose any type of flow rate regulation, therefor the flow rate delivered by such an inhalation device can be rather unreliable and hence is not suitable to be applied for proper medicine or nicotine delivery purposes. Furthermore, the document only discloses a basic principle of using a PWM control, but no teaching is given about a precise regulation of the mixing of the two atomized liquid.

US 2017/0262613 A1 discloses a medical device for dose-control, wherein the inhalation process can be controlled and supervised by a doctor.

US 2018/0289907 A1 discloses a mobile inhaler that is capable of producing a mixture to be inhaled, wherein the liquids are stored in separate reservoirs and atomized by piezoelectric atomizers.

CN 206699400 U and CN 209060231 U disclose an e-cigarette device and a portable breathing mist inhaler device, respectively. Both devices comprise an ultrasonic atomization piece for atomizing a liquid stored in a reservoir, wherein a sponge extends into the reservoir and the ultrasonic atomization piece is also in contact with the sponge.

US 2020/0016344 A1 discloses a portable device for inhalation of at least one active composition, wherein the active compositions are atomized by a piezoelectric atomizer, which allows for a production of any inhalation mixture.

US 2020/0206430 A1 discloses a multi-modal dosing device having removable and exchangeable cartridges and a piezoelectric atomizer, however the device is capable of atomizing only a content of one of the cartridges at the same time, thus mixing of the content of the different cartridges is not possible.

US 2009/0095821 A1 discloses a piezoelectric actuator control system that aims to achieve volume flow regulation based on electric current measurement. It proposes a solution to test a response of the system in the frequency domain. Therefore, a frequency sweep is performed on the piezo actuator before every usage. However, such a frequency sweep is very time-consuming and the evaluation of the signals requires a high amount of calculation capacity. The document does not disclose a way for handling other environmental effects affecting the system, e.g., temperature, external mechanical forces, viscosity of the atomized liquid, etc. On the other hand, such a frequency sweep is only representing a maximum volume flow working point of the output stage, furthermore, it is also not ensured that the frequency sweep always finds the right frequency which can cause errors in the estimation of the flow rate and thus in the delivered dosage of a medicine.

A major disadvantage of inhalation devices known in the art is that these do not allow for a precise mixing and dosage of atomized liquids, because these do not take into account the uncertainties of the flow rates of the atomized liquids. As a result, the mixing ratio and the exact dosage delivered to the user or patient can be different than the desired mixing ratio and desired dosage.

In view of the known approaches, there is a need for an inhalation device having a more precise mixing and a more accurate dosage and also a method for regulating the mixing of atomized liquids.

### DESCRIPTION OF THE INVENTION

The primary object of the invention is to provide an inhalation device, which is free of the disadvantages of prior art approaches to the greatest possible extent.

The object of the invention is to provide an inhalation device having a precise flow rate control by which an accurate mixing of atomized liquids and their delivery is ensured.

A further object of the invention is to provide an inhalation device that is easy to use and that minimizes wasting of the atomized liquids.

A still further object of the invention is to provide an inhalation device, wherein the exact dosage incorporated by the user or the patient can be monitored by high precision.

The objects of the invention can be achieved by the inhalation device according to claim 1. Preferred embodiments of the invention are defined in the dependent claims.

The main advantage of the method according to the invention compared to prior art approaches comes from the fact that it allows for a precise control of the flow rate of the atomized liquids, thus the dosage delivered by the inhalation device can be monitored in a more effective and precise manner.

It has been recognized, that piezoelectric actuators traditionally used as atomizers in inhalation devices do not provide stable and constant vibrations when facing mechanical influences. The vibrations can change as an effect of pushing a button on the inhalation device, i.e., turning on or off the device, or even during the use of the device. Unstable vibrations alter the flow rate of the atomized liquids resulting in a changing quantity of the atomized liquid. Atomizers having a vibrating mesh can have an uncertainty up to 30% of the flow rate. When two such atomizers are used to atomize two different liquids, the uncertainty of each atomizer leads to an uncertain mixing ratio, and an uncertain dosage delivered by the inhalation device. Such an uncertainty may cause under or over dosage of the atomized liquids.

The inhalation device according to the invention electronically controls the piezoelectric actuators, thus provides stable and known flow rates of the atomized liquids. The factors affecting the flow rates have been recognized and have been implemented in a preferred embodiment of the inhalation device.

A further advantage of the inhalation device according to the invention is that a user inhalation is detected, thus the inhalation device only delivers the mixture of the atomized liquids when the device is in use which on the one hand prevents wasting of the atomized liquid and on the other hand further improves the certainty of the delivered dosage. The user is thus provided with an exactly known dosage of atomized liquids according to a predetermined mixing ratio, thus the user, the patient or the doctor of the patient can monitor the use and the dosage delivered by the inhalation device. Such monitoring makes it easier for a patient, especially elderly patients to operate the device and to ensure that the required dose has been delivered.

It has also been recognized that for regulating the flow rate of a piezoelectric atomizer, state variables need to be identified that are responsible for a change in the flow rate generated by the piezoelectric atomizer. The identification and control of such state variables allow for maintaining a stable flow rate resulting in a precise control of the dosage delivered by the inhalation device.

A further advantage of the invention is that it has an easy and quick use, without a need of any kind of calibration before each use of the inhalation device. Any required flow rate can be provided any time, besides the inhalation device according to the invention ensures that the user will be provided with the exact same dosage and mixing ratio as desired.

### BRIEF DESCRIPTION OF THE DRAWINGS

Preferred embodiments of the invention are described below by way of example with reference to the following drawings, where

FIG. 1 is a preferred embodiment of a regulating electrical circuit of the inhalation device according to the invention,

FIG. 2 is a preferred regulation loop for regulating a flow rate of the inhalation device according to the invention,

FIG. 3 is a schematic drawing of an output stage current both in a load and a no-load condition when driven by a 50% duty cycle switching frequency,

FIG. 4 is a schematic drawing of a preferred open regulation loop,

FIG. 5 is another preferred embodiment of a regulating electrical circuit of the inhalation device according to the invention,

FIG. 6 is a diagram showing the effect of driving two output stages simultaneously in a configuration according to FIG. 5,

FIG. 7 is a further preferred embodiment of the inhalation device according to the invention comprising a pod,

FIG. 8 is a preferred embodiment of a boost converter stage of the regulating electric circuit,

FIG. 9 is a preferred embodiment of a current measurement stage of the regulating electric circuit,

FIG. 10 is a preferred embodiment of an output stage of the regulating electric circuit,

FIG. 11 is a preferred embodiment of a controller stage of the regulating electric circuit,

FIG. 12 shows simulation results to describe a high duty cycle behaviour of the inhalation device according to the invention,

FIG. 13 is a state diagram describing the regulation functionality of a software and a controller of the inhalation device according to the invention, and

FIGS. 14 and 15 are preferred embodiments of a pressure sensor stage of the regulating electric circuit.

### MODES FOR CARRYING OUT THE INVENTION

The invention relates to an inhalation device for delivering an atomized mixture of liquids with a regulated mixing ratio. The inhalation device comprises two reservoirs, each storing a respective liquid, wherein the liquids can be two ingredients of a medicine to be inhaled by a patient, or in other applications one of the liquids can be liquid nicotine and an other one of the liquids can be a diluent liquid. The mixing ratio of the two liquids is variable and it can be set to any ratio from 0% to 100% by a user, a doctor or a patient.

The reservoirs are preferably exchangeable and/or refillable, thus an empty reservoir can either be changed to a filled reservoir or the reservoir can be refilled with liquid. For medical applications exchangeable reservoirs are preferred over refillable reservoirs as it allows for a more precise control of the delivered dosage of the medicine. Preferably, when one of the reservoirs is empty, the inhalation device still can be used with a 0% mixing ratio corresponding to the empty reservoir.

For each reservoir the inhalation device comprises a respective piezoelectric actuator for atomizing the respective liquid, wherein the piezoelectric actuator is preferably a piezoelectric mesh actuator, i.e., a piezoelectric mesh transducer disc, having a first side and an opposite second side and through holes. Preferably, the through holes are extending between the first side and the second side in a direction of a vibration of the piezoelectric actuator, wherein ends of the through holes on the first side are in contact with the liquid, even more preferably with a liquid transferring member, and the atomized liquid is generated during vibrations at ends of the through holes on the second side. The piezoelectric actuator preferably works (vibrates) in a frequency domain of 50 kHz-3 MHZ, even more preferably in a frequency domain of 100 kHz-200 KHz.

Furthermore, for each reservoir the inhalation device comprises a respective liquid transferring member made of a capillary material, wherein the liquid transferring member is in contact with the respective liquid in the reservoir and with the respective piezoelectric actuator. The capillary material is preferably a wicking, a sponge, a foam, a filter, or any other suitable material that is capable of storing and continuously transferring liquid from the reservoir to the piezoelectric actuator.

Preferably an end of the liquid transferring member is in continuous contact with the piezoelectric actuator, even more preferably the end of the liquid transferring member is pressed to the piezoelectric actuator. By pressing the liquid transferring member to the piezoelectric actuator, the liquid transferring member continuously supplies liquid to the piezoelectric actuator in any orientation of the inhalation device.

The inhalation device further comprises a pressure sensor for detecting a user inhalation, wherein the pressure sensor is preferably arranged in a mouthpiece of the inhalation device to detect the inhalation of a user. When the user inhales, a pressure signal measured by the pressure sensor drops, and when the user stops the inhalation the pressure signal recovers to an original value, thus the pressure signal can be evaluated and used in order to determine when the inhalation device is in use and when atomization of the liquids is necessary.

The inhalation device according to the invention preferably comprises a further pressure sensor for measuring ambient pressure. In this case, the user inhalation can be detected based on a pressure difference between the two pressure sensors, i.e., when the pressure difference measured by the two pressure sensors exceeds a predetermined pressure difference limit. When the inhalation device is not in use, i.e, no user inhalation is detected, the two pressure sensors both measure the ambient pressure, thus the pressure difference is close to zero. When a user starts using the inhalation device, the pressure value measured by the pressure sensor detecting the user inhalation drops, thus the pressure difference increases.

The inhalation device according to the invention further comprises a regulating electric circuit for regulating a flow rate of the atomized liquids by controlling the piezoelectric actuators. The flow rate can be a volume flow rate, a mass flow rate and it is regulated by maintaining a stable flow rate during the use of the inhalation device, namely, when the user is inhaling or sipping the inhalation device.

The regulating electric circuit comprises a controller connected to the pressure sensor or pressure sensors, when more than one pressure sensor is used, preferably to receive inhalation data, wherein the inhalation data preferably contains data characterizing the inhalation such as a start time of the inhalation, an end time of the inhalation, a duration of the inhalation, or an intensity of the inhalation, etc. The controller connected to the pressure sensor enables atomizing only during the user inhalation.

The regulating electric circuit further comprises an output stage for each of the piezoelectric actuators, wherein each output stage comprises an inductor forming a resonant circuit with the respective piezoelectric actuator having an inherent capacitance. The inherent capacitance is preferably a parallel capacitance in respect to the inductor.

Each output stage further comprises a switch for driving the output stage by a respective output PWM signal having a duty cycle, wherein output PWM signal is generated by the controller. The switch is preferably a MOSFET or a bipolar transistor, and the switch preferably has a drain-source or a collector-emitter breakdown voltage greater than 200 V in order to avoid short circuit failure if no piezoelectric actuator is attached to the regulating electric circuit.

Each output stage is driven by a respective output PWM signal having a duty cycle and being generated by the controller, wherein the mixing ratio and the flow rates of each atomized liquids are determined by the duty cycles of the output PWM signals.

In one embodiment the regulating electric circuit preferably comprises a feedback circuit connected to the controller for regulating a state variable characterizing the flow rate of the atomized liquids based on a predetermined reference value of the state variable. The state variable is preferably a current, a voltage, a frequency of the piezoelectric actuator or a combination thereof. The predetermined reference value is preferably chosen from reference values of the state variable that are preferably stored in a look-up table in a memory of the controller.

The predetermined reference value preferably corresponds to circumstances affecting the flow rates of the atomized liquids, wherein the circumstances include at least one of the following: self-heating of the inhalation device, variations of environmental temperature, moisture, aging of the inhalation device, viscosity of the respective liquid, a material of the respective liquid transferring member, contact between the respective liquid transferring member and the respective piezoelectric actuator, mechanical forces acting up on the inhalation device. These circumstances might have a non-linear relation with the flow rate, for example the self-heating of the inhaling device results in a viscosity change of the liquid stored in the reservoirs, wherein the viscosity can also affect the flow rate. The reference values can be measured and determined once before the production of the inhalation device, and the reference values can be stored in the memory of the controller.

In a preferred embodiment of the invention, the inhalation device comprises one feedback circuit that is connected to all of the output stages to return feedback from the output stages to the controller.

In another embodiment the regulating electric circuit preferably comprises two feedback circuits, one for each piezoelectric actuators, wherein each feedback circuit is connected to the controller and to the respective output stage to return feedback from the respective output stage to the controller.

The inhalation device according to the invention preferably comprises a battery, preferably a chargeable battery, connected to the controller for supplying power to the controller.

The controller of the regulating electric circuit preferably further comprises a temperature sensor module that can track an internal temperature of the inhalation device according to the invention.

In another preferred embodiment of the invention the inhalation device preferably comprises an external temperature sensor for tracking an internal temperature of the inhalation device according to the invention and also a temperature in the vicinity of the piezoelectric actuators.

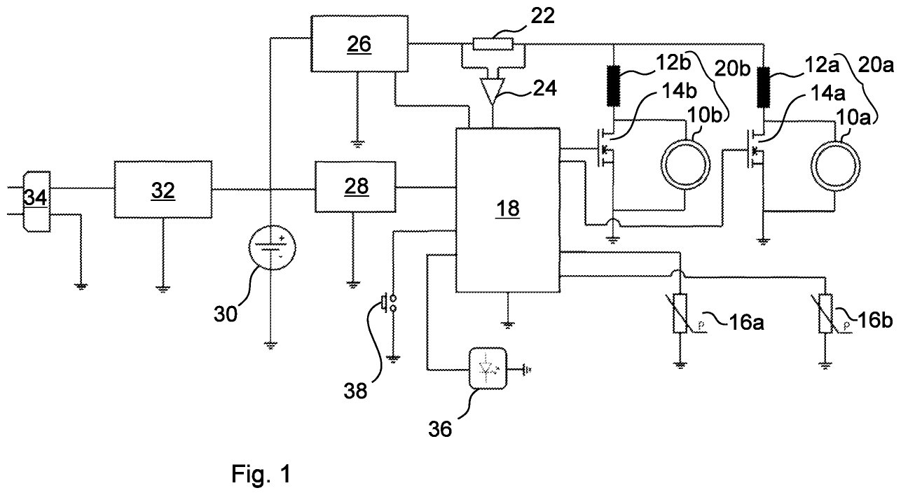

A preferred embodiment of a regulating electrical circuit of the inhalation device according to the invention is shown in FIG. 1. The regulating electrical circuit according to this embodiment is based on an embedded solution for driving two piezoelectric actuators 10a, 10b, wherein the piezoelectric actuators 10a, 10b are piezoelectric mesh transducer discs having through holes and their vibrations are preferably in a frequency domain of 50 KHz-3 MHZ, even more preferably in a frequency domain of 100 KHz-200 kHz. Atomization of a liquid can be achieved through the through holes of the piezoelectric actuators 10a, 10b.

The regulating electrical circuit according to FIG. 1 comprises a controller 18, preferably a microcontroller, and preferably a software is loaded to the controller 18.

The regulating electrical circuit comprises two output stages 20a, 20b, one for each of the piezoelectric actuators 10a, 10b, wherein each output stage 20a, 20b comprises an inductor 12a, 12b forming a resonant circuit, preferably a resonant LC circuit, with the respective piezoelectric actuator 10a, 10b having an inherent capacitance being a parallel capacitance in the resonant circuit in respect to the inductor 12a, 12b. Each output stage 20a, 20b further comprises a switch 14a, 14b controlled by the controller 18, wherein each switch 14a, 14b enables atomizing only during a user inhalation. Preferably, the switches 14a, 14b are directly driven by the controller 18. Preferably, each switch 14a, 14b can be a MOSFET or a bipolar transistor. Preferably, the switches 14a, 14b have a collector-emitter or drain-source breakdown voltage greater than 200 V in order to avoid short circuit failure when a piezoelectric actuator 10a, 10b is not attached to the regulating electrical circuit.

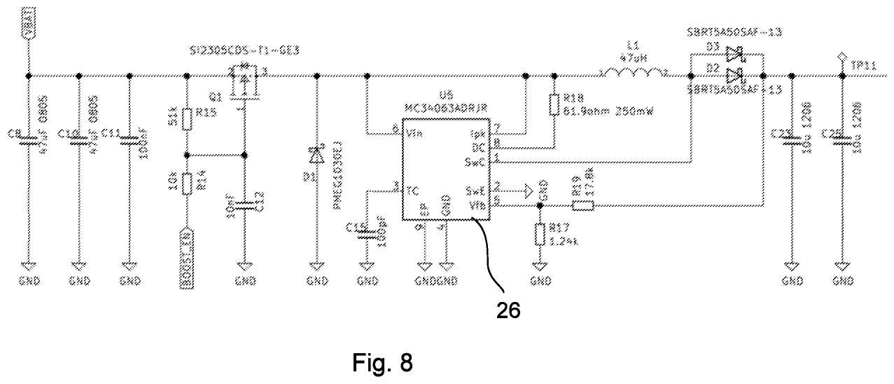

The regulating electrical circuit according to FIG. 1 further comprises a boost converter 26, from which a supply voltage or the output stages 20a, 20b can be derived. Depending on a required power to be delivered to the output stages 20a, 20b, the supply voltage is usually between 15 V-25 V. The boost converter 26 is preferably a part of a boost converting stage of the regulating electrical circuit. A preferred implementation of the boost converting stage is shown in FIG. 8.

The controller 18 is using an output PWM (Pulse Width Modulation) signal for controlling the output stages 20a, 20b. The output PWM signal has a switching frequency that is in the vicinity of a self-resonance frequency of the piezoelectric actuators 10a, 10b, but it is not exactly the same frequency value. This is due to the fact, that circuit components of the output stages 20a, 20b are tuning the piezoelectric actuators 10a, 10b from its self-resonance frequency to another value, where a maximum flow rate can be realized with a minimal power loss in the regulating electrical circuit.

Considering one of the output stages 20a, 20b, when the output PWM signal is activated, the respective LC resonant circuit starts to oscillate on the switching frequency. This oscillation is transformed into an oscillating current on the series equivalent resistance of the piezoelectric actuator 10a, 10b. The oscillating current is further transformed into a mechanical excitation having a same frequency as of the switching frequency that eventually turns on the piezoelectric actuator 10a, 10b and it starts to atomize the respective liquid of the piezoelectric actuator 10a, 10b in the inhalation device.

In order to continuously regulate the flow rate of the atomized liquid, the controller 18 is continuously monitoring the current delivered to the output stages 20a, 20b with a feedback circuit, preferably with a current feedback circuit, formed by a shunt resistor 22 (that can be a discrete resistor, a variable resistor or a potentiometer) and an amplifier 24, preferably a current-sense amplifier or an operational amplifier. The amplifier 24 provides an analogue output signal towards the controller 18 that is directly proportional to a value of electric current flowing to the output stages 20a, 20b. Therefore, the controller 18 is able to perform current regulation on the output stages 20a, 20b. The control parameter of the current regulation is a duty cycle (a pulse width) of the output PWM signal. Larger pulse width applied to the switches 14a, 14b will result in more supplied electric current to the output stages 20a, 20b and proportionally it will also deliver more power to the piezoelectric actuators 10a, 10b. The connection between the supplied electric current to an output stage 20a, 20b and the flow rate of that output stage 20a, 20b is determined with a series of measurements and saved in a memory, preferably a non-volatile memory of the controller 18.

The inhalation device according to the invention can be supplied with a battery 30, preferably a rechargeable battery 30 such as a lithium battery or a lead-acid battery. Preferably, the controller 18 is directly supplied by a switched-mode power supply or a linear regulator 28, that sets a nominal supply voltage of 3.3 V for the controller 18.

The rechargeable battery 30 can be recharged by a battery charger module 32. The state of charge is continuously monitored by the controller 18 in the preferred embodiment according to FIG. 1, and preferably it is also indicated for a user on an optical terminal 36, wherein the optical terminal 36 is preferably a coloured indication light or a display. The battery 30 can be recharged through a connector 34. The connector 34 can be any type of connector, however for the inhalation device according to the invention which is a small-sized hand-held device, a micro-USB type of connector is preferred. The user can plug an appropriate type of cable into the connector 34. The charging can be completed by any type of 5 V voltage supply units, e.g., a laptop USB connector or a wall-socket plug charger device having a required cable receptacle (usually USB type A).

The inhalation device according to FIG. 1 comprises two pressures sensors 16a, 16b. Pressure sensor 16a is preferably arranged in the inhalation device in a specific location, where it can sense a pressure change due to a user inhalation, i.e., in a mouthpiece of the inhalation device. The other pressure sensor 16b preferably arranged at a different location and it preferably measures an ambient pressure. When the user starts to inhale or sip a front end (e.g., a mouthpiece) of the inhalation device, then a signal of pressure sensor 16a drops. This can be used to determine a start time and/or an end time and/or a duration of the user inhalation. Preferably, the intensity of the user inhalation can also be determined based on a signal measured by the pressure sensor 16a.

By using two pressure sensors 16a, 16b, the start time and/or the end time and/or the duration of the user inhalation can also be determined based on a pressure difference between the two pressure sensors 16a, 16b. The controller 18 is preferably continuously monitoring the measured values of the pressure sensors 16a, 16b and calculates a difference. When the pressure difference between the two sensors 16a, 16b exceeds a predetermined pressure difference limit preferably for a given amount of time (being a glitch filter time), the controller 18 predicts that a user wants to use the inhalation device. Therefore, the controller 18 starts the driving the output stages 20a, 20b according to a predetermined mixing ratio and the atomization of the two liquids placed in respective reservoirs by the piezoelectric actuators 10a, 10b begins. The controller 18 keeps monitoring the pressure difference between the two pressure sensors 16a, 16b. As long as the user inhales or is sipping the inhalation device, the output stages 20a, 20b are driven. When the user stops the inhalation or the sipping, the pressure difference goes below the predetermined pressure difference limit (which is defined as a switching off value for the output stages 20a, 20b), then the controller 18 shuts down the output stages 20a, 20b. The pressure sensors 16a, 16b can be either digital or analogue sensors.

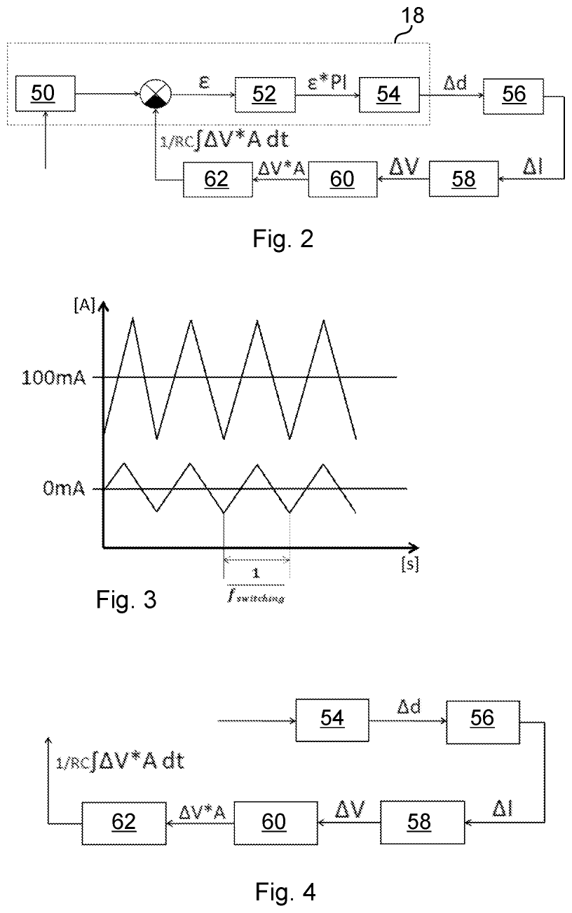

In order to exactly monitor and regulate the flow rate of the inhalation device, preferably a regulation loop according to FIG. 2 can be implemented.

A hand-held inhalation device cannot comprise a flow rate sensor as a usual size of a flow rate sensor is normally larger than that of the inhalation device. In the lack of a flow rate sensor or a volume flow sensor, according to the invention, a state-variable has been selected that reflects the behaviour of the flow rates to be observed. A current, a voltage, a frequency of the piezoelectric actuator 10a, 10b can be suitable as a state variable. A combination of the aforementioned potential state variables can also be used as a state variable.

In FIG. 2, a preferred regulation loop is presented, wherein the state variable is the current of the piezoelectric actuator 10a, 10b, preferably a load current of the output stage 20a, 20b. The piezoelectric actuator 10a, 10b is forming a complex system with its environment so it is not possible to simply describe it with a series of fundamental equations taken from basic physical correlations. To overcome this problem, a set of consecutive measurements are to be prepared to characterize the behaviour of the load current in correlation with the flow rates generated by the piezoelectric actuators 10a, 10b during several environmental circumstances such as self-heating, environmental temperature variation, moisture, ageing of the inhalation device, viscosity of the liquid used for atomization, a material of the respective liquid transferring member, contact between the respective liquid transferring member and the respective piezoelectric actuator 10a, 10b, and mechanical forces acting up on the inhalation device (i.e., the effect of mechanical forces applied on a housing of the inhalation device).

The above environmental circumstances are not independent from each other. For example, the temperature change of the piezoelectric actuators 10a, 10b can change the temperature of the liquid, which can further change the viscosity of the liquid, wherein the viscosity of the liquid also affects the flow rate greatly. These dependencies can make the flow rate regulation complex and non-trivial.

The results of such measurements, as being reference values 50 for the regulation loop, are collected and included in an array or in a database, and are preferably stored in a memory of the controller 18 of the inhalation device in a form of a look-up table. For regulation purposes, the controller 18 will choose a suitable reference value 50 from the look-up table based on the abovementioned environmental circumstances, e.g., if the controller 18 is aware what type of liquid is used for atomization and a viscosity value corresponding to that specific type of liquid. Besides getting the reference values 50 from the look-up table, the controller 18 could also interpret a correct reference value 50 by using predetermined functions, e.g., if there are a few measured reference values 50 saved in the memory for different operating temperatures, than the correct reference value 50 for the actual operating temperature can be determined for example by interpolation. Therefore, the controller 18 is able to choose and use the correct reference value 50 that most precisely reflects the behaviour of the flow rate with that specific type of inhalator liquid. The controller 18 can use further data such as a temperature data determined by a temperature sensor module of the controller or by an external temperature sensor connected to the controller for choosing the correct reference value 50.

The regulation loop according to FIG. 2 shows that the controller 18 compares a measured state variable with a respective reference value 50 for that state variable. For the comparison, the measured state variable that is usually an analogue signal is digitalized by the controller 18. In the preferred embodiment of the regulation loop, an analogue voltage signal is digitalized by the controller 18, and then the selected predetermined reference value is deducted from it. This results in a difference value denoted by ε, that can be interpreted as an error value. The difference value ε goes through a transfer function of a PI compensator 52 that is preferably coded in the software of the controller 18. The PI compensator 52 is responsible for the stability and precision of the regulation loop. A proportional constant P and an integral constant I of the PI compensator 52, and thus the behaviour of the regulation electric circuit is investigated by injecting a step function to the loop in an open loop mode as it is described in more detail in connection with FIG. 4.

An output of the PI compensator 52 denoted by ε*PI is preferably directed into a timer module 54 of the controller 18 to generate an output PWM signal. The timer module 54 of the controller 18 preferably outputs a duty cycle state variable Δd that defines the duty cycle of the output PWM signal. When the controller 18 or the software of the controller 18 refreshes the timer module 54, the timer module 54 registers a new value, and the output of the timer module 54 will change to an output PWM signal having a square wave shape and a duty cycle. The output PWM signal is then directed into a block 56 of the output stages 20a, 20b to drive the output stages 20a, 20b.

The regulation loop according to FIG. 2 also shows that any type of disturbance of the current (denoted by ΔI) of the output stages 20a, 20b is multiplied by a shunt resistance gain 58 of the shunt resistor 22, denoted by R; then it is further multiplied by a shunt amplifier gain 60 of the amplifier 24, denoted by A. This way, the current of the output stage 20a, 20b is transformed into an amplified voltage value, however it has a very specific waveform that can be observed when driving a resonant output stage 20a, 20b with piezoelectric actuators 10a, 10b as the loads. This waveform cannot be sampled directly and effectively with an AD converter of the controller 18 for further processing. Instead, an integrator 62 has to be implemented as a next stage in the regulation loop that smooths out the switching harmonics from the load current waveform so that the controller 18 is able to continuously interpret its value.

FIG. 3 schematically shows output stage currents in load and in no-load conditions when driven by a 50% duty cycle switching frequency.

In a no-load condition the piezoelectric actuators 10a, 10b represent an infinite impedance or it is a condition, wherein the piezoelectric actuators 10a, 10b are not connected to the output stages 20a, 20b. In no-load condition a triangular current waveform can be seen that oscillates around 0 mA. If it is integrated (and dissipation losses are neglected), the integrated signal equals approximately zero. The triangular waveform seen in FIG. 3 is an effect of the inductors 12a, 12b being periodically magnetized and demagnetized within one period of the switching frequency of the output stages 20a, 20b.

However, in load conditions, when the piezoelectric actuators 10a, 10b are working in the output stage 20a, 20b and a flow of atomized liquid is generated, then the current still has a triangular waveform, but it is shifted upwards, in the example according to FIG. 3, to around 100 mA. When this signal is integrated, it will result in a constant current value greater than zero. If a small unavoidable power dissipation is disregarded on the switches 14a, 14b of the output stages 20a, 20b, then by multiplying the integrated current signal in load condition with a constant boost converter output voltage, then the resulting power value is a power value that the piezoelectric actuators 10a, 10b are using to generate the flow of the atomized liquids, and therefore this power value is directly proportional to the flow rate of the atomized liquids. Based on the above considerations, the current of the output stages 20a, 20b is a suitable candidate for being a state variable that can be used for a precise regulation of the flow rates of the inhalation device according to the invention.

FIG. 4 shows a further analysis of the regulation loop, comprising a step of quantizing an analogue signal with a linear regulator 28 (implemented as an AD converter) of the controller 18. As a rule of thumb, the sampling frequency of the AD converter shall be ten times higher than the crossover frequency of the regulation loop. E.g., if the switching frequency of the piezoelectric actuators 10a, 10b is 129 kHz, then the crossover frequency of the regulation loop shall be less than 10% of the switching frequency, hence the regulation loop is to be designed with a crossover frequency of 12.9 kHz or less, while a sampling frequency of the AD converter is not to be less than 129 kHz, otherwise the regulation can be unstable under certain conditions. FIG. 4 shows the abovementioned open loop investigation of the behaviour of the regulation electric circuit for designing the PI compensator 52.

For outputting the duty cycle state variable Δd, the controller 18 can use its timer module 54 to generate an output PWM signal. When the controller 18 or the software of the controller 18 refreshes the timer module 54, the timer module 54 registers a new value, and the output of the timer module 54 will change to an output PWM signal having a square wave shape and a duty cycle. So, to investigate the step response of the regulation electric circuit, the software should set the timer module 54 to register a modified duty cycle of the output PWM signal having a new value for the duty cycle and this way the behaviour of the signal as aforementioned at the output of the integrator 62 can be observed.

It can be seen that the output of the timer module 54 of the controller 18 directly goes to the block 56 of the output stage 20a, 20b, thus it can be concluded that the change in duty cycle (Δd) directly regulates an output state variable ΔI of the output stage 20a, 20b. With this, the regulation loop is complete. It is to be mentioned, that the above discussed regulation loop can also be designed with analogue discrete or integrated components, omitting the need for the resources of the controller 18 to directly take part in the regulation. With this alternative method, a type-1 or type-2 compensator circuit built from an operation amplifier instead of the PI compensator block 52 can also be used. The output PWM signal can be substituted with a sawtooth generator and a comparator circuit such as it can be found in any switched mode DC/DC converters using a voltage-mode control. Additionally, the type-1 or type-2 compensator circuit and the output PWM signal can be substituted by a switched-mode DC/DC converter having a voltage-mode control and having a direct access for a designer to design the compensation network built in the switched-mode DC/DC converter.

An FPGA or a CLPD module can also be used instead of a microcontroller for designing the regulation loop components.

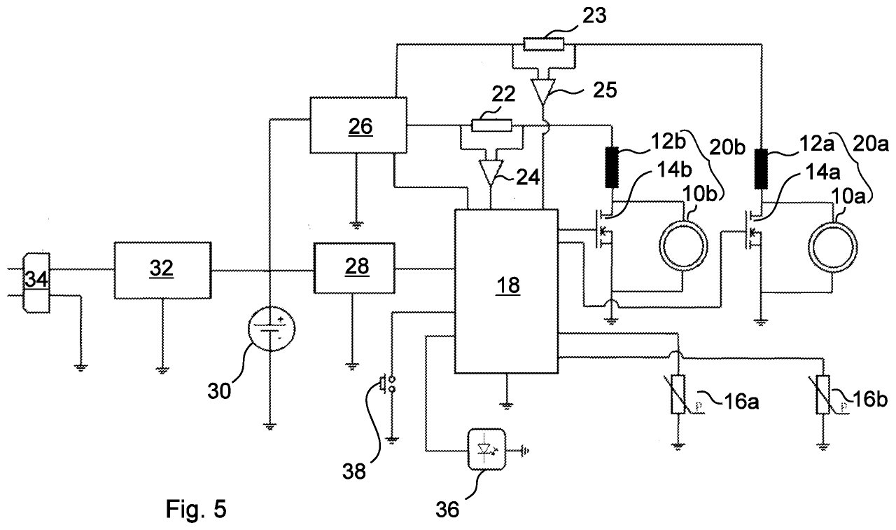

FIG. 5 shows an other preferred embodiment of a regulating electrical circuit of the inhalation device according to the invention, similar to the preferred embodiment according to FIG. 1. The difference between the embodiment according to FIGS. 1 and 5 lies in the feedback circuit, the other parameters and characteristics of the two embodiments can be the same.

The embodiment according to FIG. 5 comprises two feedback circuits, i.e., one feedback circuit for each output stage 20a, 20b, thus the output stages 20a, 20b can be driven and can be activated separately, as individual feedback is received from each piezoelectric actuators 10a, 10b.

For example, according to the embodiment of FIG. 1, if the two output stages 20a, 20b are operated with a base time width (duty cycle) of 50 milliseconds and a mixing ratio of 80%, then one of the output stages 20a, 20b can be operated only for 20% of this base time width of 50 milliseconds, thus, for 10 milliseconds, and after this, the other one of the output stages 20a, 20b can be operated for 80% of the base time width, thus, for 40 milliseconds. After this, the base time width starts again. This way a mixing of the flows (preferably the volume flows) of two different liquids atomized by the piezoelectric actuators 10a, 10b can be precisely realized with the help of the abovementioned flow rate regulation loop according to FIGS. 2 and 4. The mixing ratio of the two flow rates can be adjusted by a user via a button 38, wherein any mixing ratio between 0% and 100% can be selected.

The actual mixing ratio is preferably communicated to the user via an optical terminal 36 such as a LED array, or an LCD display.

The inhalation device according to the invention makes it possible to mix two ingredients of a medicine with a precise mixing ratio, or in other applications liquid nicotine could be to use for one of the output stages 20a, 20b and a diluent liquid can be used for the other one of the output stages 20a, 20b, wherein the user can set any mixing ratio for the two liquids. For example, the user can start from 100% nicotine liquid and 0% diluent liquid at the beginning, and later the mixing ratio can be adjusted to a different value which allocate less amount of nicotine in the user's body within the same time. This feature of the inhalation device can be used to make the user progressively reduce and finally abandon the need for a daily nicotine intake by continuously setting a lower mixing ratio for the nicotine. The button 38 can be implemented in a form of a physical push-button or of a capacitive sensor.

The switching frequency of one of the output stages 20a, 20b is preferably determined early in the production of the inhalation device according to the invention by using a shunt resistor 22 and an amplifier 24. As it has been discussed in correlation with FIG. 1, the shunt resistor 22 can be a discrete resistor, a variable resistor or a potentiometer, and the amplifier 24 can be a current-sense amplifier or an operational amplifier.

The switching frequency preferably can be determined by the following way. First, the controller 18 starts to drive one of the output stages 20a, 20b on a switching frequency of e.g., 125 kHz for a piezoelectric actuator 10a, 10b, which has a nominal self-resonance frequency of 135 kHz. The controller 18 is continuously monitoring a current supplied to the driven output stage 20a, 20b by the shunt resistor 22 and the amplifier 24. Then an upwards frequency sweep is started until e.g., 150 KHz with a fine sweeping resolution of e.g., 300 Hz. During this process the current supplied to that output stage 20a, 20b is continuously registered. When the sweep is complete, the controller 18 selects a specific frequency where the highest current supplied to that output stage 20a, 20b has been measured. This frequency also corresponds to reaching the highest flow rate value for that output stage 20a, 20b. This specific frequency is preferably saved in a memory, even more preferably in a non-volatile memory of the controller 18 and when the inhalation device is in use, this frequency will be used as a general switching frequency of that output stage 20a, 20b. The switching frequency obtained by the above process corresponds to the highest flow rate versus internal power loss ratio, thus contributes to the effective operation of the inhalation device according to the invention.

As for FIG. 5, and additional feedback circuit is implemented, preferably for a current feedback measurement comprising a shunt resistor 23 and an amplifier 25, wherein the shunt resistor 23 is preferably a discrete resistor, a variable resistor or a potentiometer, and the amplifier 25 is preferably a current sense amplifier or an operational amplifier. The topology according to FIG. 5 provides separate voltage supply to the two output stages 20a, 20b with separate feedback circuits, preferably for separate current feedback measurements. This topology—compared to the topology shown in FIG. 1—allows to drive and regulate the two output stages 20a, 20b simultaneously and independently.

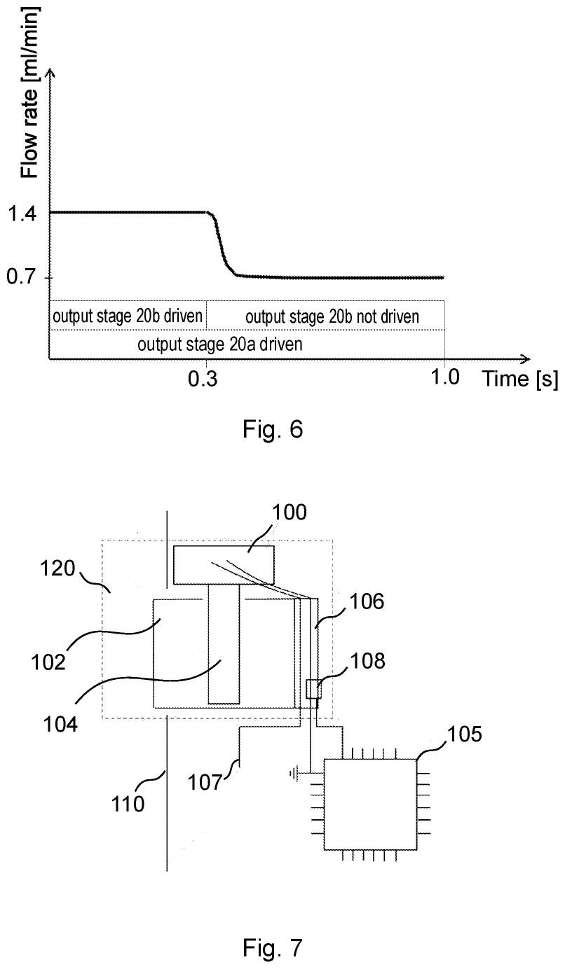

According to the preferred embodiment of FIG. 5, it is possible to atomize different liquids by the piezoelectric actuators 10a, 10b at the same time. As a result, the flow rates of the different atomized liquids are added together in the same time frame. FIG. 6 shows an example about the additional benefits the topology according to FIG. 5 can implement.

According to FIG. 6, output stage 20a is driven continuously for 1 second, while output stage 20b is driven only for 0.3 second in the same time frame, resulting in higher volume flows when both output stages 20a, 20b are driven.

FIG. 7 shows a further preferred implementation of the flow rate regulation, wherein the inhalation device according to the invention preferably comprises a pod 120 comprising a piezoelectric actuator 100, a reservoir 102 for storing a liquid, a liquid transferring member 104, a printed circuit board 106 and an external memory device 108, wherein the liquid transferring member 104 is made of a capillary material and is in contact with the liquid in the reservoir 102 and with the piezoelectric actuator 100.

One realization of the flow rate regulation could be to implement an external memory device of the pod 120 of the device. The external memory device 108 can hold information on the specific behaviour of the pod 120 that would be preliminary tested in a factory by an open loop investigation method described above in connection with FIG. 4.

This way, instead of a microcontroller an FPGA or a CLPD or an analogue compensation loop could acquire information from the external memory device 108 of the pod 120 about the behaviour of reference parameters such as a temperature, a type of liquid stored in the reservoir 102, ageing, load current, etc.

The pod 120 preferably can be attached to a housing 110 of the inhalation device so that the controller 18, FPGA, CLPD, or analogue circuit 105 can access to information from the external memory device 108. The external memory device 108 could be any type of memory device, for example a “One-Wire” device that can use a voltage supply net 107 for communication as well.

Furthermore, the pod 120 is preferably exchangeable and its reservoir 102 is preferably exchangeable and/or refillable.

Examples for the implementation of the regulating electric circuit are shown in FIGS. 8-15.

FIG. 8 shows an example of a boost converter stage comprising a boost converter 26 of the regulating electric circuit of the inhalation device according to the invention. The values and data indicated in FIG. 8 serve just as an example for implementing the boost converter stage.

The boost converter stage can use a battery 30 or any other voltage source as an input voltage. In this exemplary embodiment the circuit of the boost converter stage is designed so that it can power a handheld inhalation device with a LiPo battery of 3.7 V nominal voltage. Piezoelectric actuators 10a, 10b typically consume a power of 2 W when driven by a signal having a typical of 80 V peak-to-peak voltage. In this range of power requirement, a usual output voltage requirement of the boost converter stage is 20 V. Only a few ICs are capable of maintaining such a high duty cycle to maintain the output power of the boost converter 26. In this exemplary embodiment the IC MC34063 is used as a boost converter 26. The IC of the boost converter 26 is already implementing an internal high-side switch, therefore only diodes D2, D3 are needed to complete this stage.

A quiescent current consumption of this IC is too much for a battery-powered application, therefore a separate enabling switch Q1 is implemented between the battery input denoted by VBAT and the boost converter 26. The switch Q1 is controlled by the controller 18 (preferably implemented as a microcontroller) in a way that when a piezoelectric actuator 10a, 10b does not need to be powered, the switch Q1 is off, and thus a leakage current consumption of the boost converter stage is negligible. Diode D1 is implemented in a way that when the switch Q1 is off, the remaining energy in inductor L1 can be circulated in a low-resistance path to avoid voltage overshoots on the inputs of the IC of the boost converter 26. Inductor L1 must have an inductance value great enough to avoid discontinuous conduction mode of the boost converter control, to minimize voltage stresses, EMC emission and power dissipation on the boost converter stage components. Resistors R17 and R19 are forming a voltage divider to set an output voltage of the boost converter 26 to 19.5V.

The ratio of the resistors can be calculated by the following equation:

Vₒᵤₜₚᵤₜ/(R17+R19)*R17=V_{reference},

wherein V_{reference} is an internal reference voltage of a Vfb pin of the boost converter 26.

The switching frequency of the boost converter 26 can be set by capacitor C15. For the boost converter 26 according to the example of FIG. 8, the highest switching frequency of 120 KHz is used by applying a 100 pF capacitance for capacitor C15.

In the boost converter stage, capacitors C23 and C25 are responsible to smooth the switching ripple of the boost converter 26. With an output power of 2 W and an input voltage of discharged battery of 3.4 V and a boost converter efficiency of n=0.7, the input average current requirement can be 2 W/3.4V*1/n=840 mA. By having a 30% ripple current design, a current peak of 840 mA*1.3=1092 mA is to be expected. The voltage ripple originating from the current ripple can and needs to be smoothed by the capacitors C23 and C25.

If multi-layer ceramic capacitors (MLCC capacitors) are used, these should have a voltage rating of at least 50 V DC so the 19.5 V output voltage derating does not have significant effect on their capacitance.

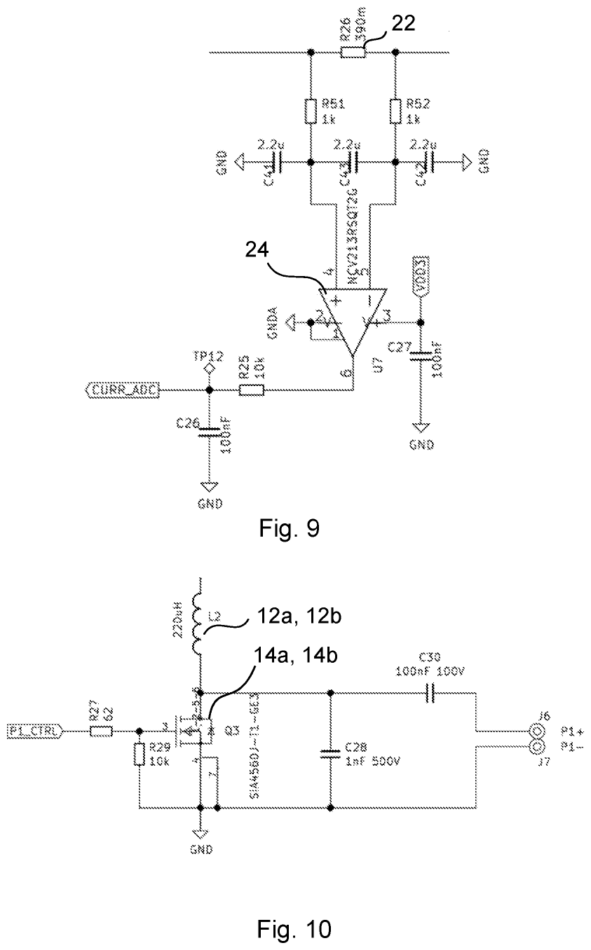

FIG. 9 shows an example of a current measurement stage of the regulating electric circuit of the inhalation device according to the invention, the current measurement stage preferably being a part of the feedback circuit. The current measurement stage comprises a shunt resistor 22 (denoted by R26) and an amplifier 24 (being an operational amplifier and denoted by U7). The values and data indicated in FIG. 9 serve just as an example for implementing a preferred current measurement stage.

The current measurement stage can be implemented either as a high side or low side measurement. In the example shown in FIG. 9, high side measurement is implemented. A current from the piezoelectric actuators 10a, 10b is flowing through resistor R26 being the shunt resistor 22. If a nominal output power of 2 W and 19.5 V output voltage is assumed for the piezoelectric actuators 10a, 10b, then an average current consumption of the piezoelectric actuators 10a, 10b is 2 W/19.5V=100 mA. Resistor R26 and a gain of the operational amplifier U7 are preferably designed in a way that an output voltage of the operational amplifier U7 will not swing to the 3.3 V supply voltage at maximum current consumption of the piezoelectric actuators 10a, 10b. It is advisable to leave a margin of more than 1 V on the output of the operational amplifier U7 at the maximum current consumption of the piezoelectric actuators 10a, 10b. In FIG. 9, the operational amplifier U7 was chosen to be a current sense amplifier NCV213RSQT2G having an internal gain of 50. A resistor value of 0.390 ohms was chosen for the shunt resistor 22, thus the output voltage of the operational amplifier U7 at the average current consumption of 100 mA of the piezoelectric actuators 10a, 10b is 100 mA*0.390 ohms*50=1.95 V. The remaining margin towards the supply voltage of 3.3 V can be used for overcurrent and short-circuit detection functions.

The current measurement stage is filtered both at an input and an output of the current measurement stage with a cut-off frequency of 500 Hz against both common mode and differential mode noises.

The filtered signal is then forwarded to an analogue/digital converter input of the controller 18 to digitalize its value and to use it for regulation or overcurrent detection by the controller 18. It is important to be noted, that without filtering the input, the triangular current waveform having negative components would be directly applied to the input of the current sense amplifier. If the current sense amplifier is not using a voltage shifting reference in that case, the output current would be modulated and a high ratio of error would be present on the output voltage signal, because in the negative phases of the input current, the current sense amplifier would constantly output an output voltage close to zero as it cannot shift beneath zero.

FIG. 10 shows an example of an output stage 20a, 20b of the regulating electric circuit of the inhalation device according to the invention. The output stage 20a, 20b comprises inductor 12a, 12b (denoted by L2 in FIG. 10) and output connectors (denoted by J6 and J7) for connecting a piezoelectric actuator 10a, 10b. The values and data indicated in FIG. 10 serve just as an example for implementing an output stage 20a, 20b.

Preferably, each output stage 20a, 20b is fed by the 19.5 V voltage supply (see description of the boost converter stage) through inductor L2. Each output stage 20a, 20b is forming a resonant circuit with the parallel inherent capacitance of the piezoelectric actuator 10a, 10b and the capacitor C28. The capacitor C28 is used in order to avoid voltage overshoots when a piezoelectric actuator 10a, 10b is not connected to the output connectors J6 and J7. C30 is implemented so that the piezoelectric actuator 10a can be protected against the constant DC voltage of 19.5V.

Usually, piezoelectric actuators 10a, 10b are rated for a maximum of 12V DC voltage and stressing with voltages above this limit might reduce the lifetime of the piezoelectric actuators 10a, 10b, as safe operation is not guaranteed by the suppliers in this regime. The inductor L2 (as an implementation of inductor 12a, 12b) can be roughly sized based on the following. A resonance frequency of the resonant circuit is formed by L2, C28 and the parallel inherent capacitance of the piezoelectric actuator 10a, 10b shall be in the vicinity of a series resonance frequency of the piezoelectric actuator 10a, 10b divided by two. For a piezoelectric actuator 10a, 10b having a parallel capacitance of 3.3 nF and a series resonance frequency of 150 kHz, and starting with a value of 1 nF for C28, an inductor value is calculated as follows: 0.5/[4π2*(1 nF+3.3 nF)*150 kHz2]=L. Therefore, solving the equation results in an inductance value of 200 pH. It is preferred to choose an inductor with a ferrite or another better core material to avoid high core losses originating from the high current ripples in the inductor. Transistor Q3 (as an implementation of switch 14a, 14b) is be chosen so that its drain-source maximum voltage should be greater than 150 V to avoid a breakdown when the output stage 20a, 20b is active but no piezoelectric actuator 10a, 10b is connected. The Gate of the transistor Q3 is to be controlled by the controller 18. In this example, transistor SIA456DJ was chosen as the transistor Q3 due to its extremely low gate-source threshold voltage that fits nicely to the requirements of the 3.3V driving voltage from the controller 18.

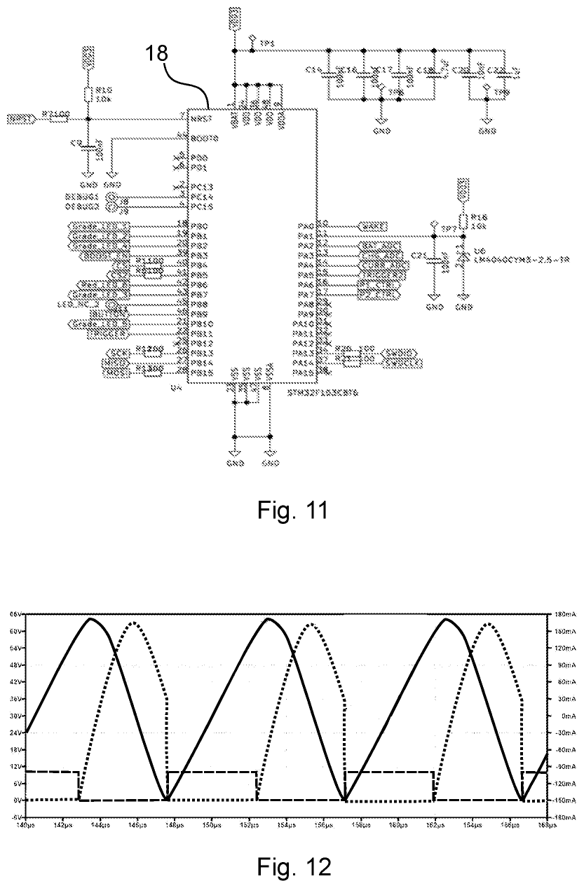

FIG. 11 shows an example of a controller stage of the regulating electric circuit of the inhalation device according to the invention. The controller stage comprises controller 18. The values and data indicated in FIG. 11 serve just as an example for implementing a controller stage.

The controller stage is responsible for controlling the output stages 20a, 20b, measuring current of the piezoelectric actuators 10a, 10b, taking inputs from a user and reading pressure sensor values. The embodiment according to FIG. 11 shows a microcontroller STM32F103C8T6 as a controller 18, but other type of controllers or microcontrollers can also be used. The controller 18 according to this embodiment is using an external 2.5 V reference voltage of U6 to avoid situations when a precise analogue measurement is needed but the voltage supply of 3.3 V drops below its nominal output voltage due to a low battery voltage level. In this example, the controller 18 is in the proximity of the output stages 20a, 20b, therefore a temperature sensor value in the controller 18 can be directly used for output current adjustment, as it was described in the previous sections. A temperature change of the output stage 20a, 20b and the piezoelectric actuator 10a, 10b is proportional with the current consumption and the flow rate of the atomized liquid. As the microcontroller STM32F103C8T6 does not have a separate PID periphery, therefore a PID control scheme is coded in a software running on the microcontroller.

When the controller 18 detects that a pressure difference value between the two external pressure sensors 16a, 16b exceeds 1000 Pa for a predetermined time of, for example, 10 ms, then the controller 18 first switches to a short-circuit detection task, which activates the output stages 20a, 20b with a 5% duty cycle, measures the current consumption, and if the consumed value is over a predetermined value of 60 mA, then the controller 18 or the software loaded onto the controller 18 jumps into error mode, and signals the user that there is a short circuit at the contacts of the piezoelectric actuators 10a, 10b (see FIG. 13 for more details). This can happen when a surface of the inhalation device has been cleaned with water but not dried completely. The applied low duty cycle ensures that the short-circuit current will not cause excessive power losses in the boost converter stage and in the output stages 20a, 20b. Furthermore, the power consumption of the piezoelectric actuators 10a, 10b with a duty cycle of 5% is only a small fraction of the nominal power of 2 W, which makes it easy for the controller 18 to detect short-circuit situations. If there is no short-circuit, the software of the controller 18 switches to the PID regulation task, and starts to regulate the piezoelectric actuators 10a, 10b to a predetermined flow rate for example that of 0.7 ml/min.

The output stage has two different transfer functions according to the duty cycle. FIG. 12 shows simulation results to describe the behaviour when the duty cycle is too high (e.g., above 60%). In FIG. 12 dashed lines denote a signal corresponding to a current of the switch 14a, 14b (e.g., a gate current when the switch 14a, 14b is implemented as a MOSFET) driven by the output PWM signal, dotted lines denote a signal corresponding to a voltage of the piezoelectric actuators 10a, 10b, and continuous lines correspond to a current of the inductors 12a, 12b.

When the output stages 20a, 20b are not driven (the current of the switches 14a, 14b is zero) the current of the inductors 12a, 12b decreases. The peak of the voltage of the piezoelectric actuators 10a, 10b lags behind the current of the inductors 12a, 12b by a phase difference of 90°.

In cases when the duty cycle is too high, there is no time for the output capacitance (C28 & inherent parallel capacitance of the piezoelectric actuators 10a, 10b) to bypass the stored energy to the inductor L1 before the output stages 20a, 20b are being activated again by the output PWM signal, and the remaining energy is immediately dissipated on the parasitic resistive elements in the circuit as it can be seen e.g., at 157 us in FIG. 12, showing that the voltage of the piezoelectric actuators 10a, 10b drops to zero when the output stages 20a, 20b are activated and the current of the switches 14a, 14b is no longer zero.

This type of behaviour must be avoided, which gives a constrain to the regulation loop to do not go over approximately 60% duty cycle and preferably it is hardcoded in the software to not allow a duty cycle control signal greater than 60% to the output stages 20a, 20b. This means, that atomizing a liquid with a viscosity of 1 cP, e.g., water, with the described mechanical and electronical configuration, a 60% duty cycle can create a flow rate of 0.85 ml/min for that specific type of liquid by using a piezo actuator FBWH-13.8Mmo-BK-IT1 from Icarus as a piezoelectric actuator 10a, 10b.

During the regulation of the piezoelectric actuator 10a, 10b, the controller 18 is preferably using a look-up table and extrapolate between discrete stored values, if necessary, to match a reference value 50 (e.g., a reference current value) with the actual mechanical design of the inhalation device, the viscosity of the liquids and the temperature. A state-diagram of FIG. 13 describes the regulation functionality of the controller 18 or the software loaded onto the controller 18.

The controller 18 can return from a piezo regulation state 74 to an idle state 70 by two different ways. Either a small enough pressure difference is detected between the two pressure sensors 16a, 16b, or a 5-second time window has passed. The second returning method is a safety feature implemented against any failure that would lead to an unintentionally long piezo regulation state 74.

As it has been described previously the controller 18 leaves its idle state 70 if the pressure sensors 16a, 16b indicate a user inhalation. Then, the controller 18 runs an overcurrent test 72 to make sure that the piezoelectric actuators 10a, 10b are connected. If no overcurrent has been detected, then either it enters the piezo regulation state and thus piezo regulation 74 is started, or the controller 18 returns to its idle state 70, if a low pressure threshold is reached or the 5-second window has passed. If an overcurrent has been detected, then the inhalation device indicates an overcurrent error 76, and 5 seconds later the controller 18 returns into its idle state 70.

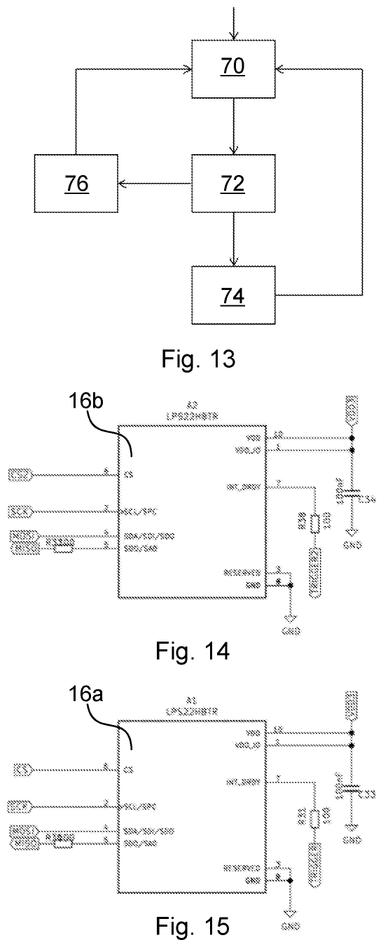

FIGS. 14-15 show examples of a pressure sensor stage of the regulating electric circuit of the inhalation device according to the invention. The pressure sensor stage comprises pressure sensors 16a, 16b. The values and data indicated in FIGS. 14-15 serve just as examples for implementing a pressure sensor stage.

Each pressure sensor stage comprising an LPS22 absolute pressure sensor IC. A surface of the pressure sensors 16a, 16b is a metal plate that is directly registering pressure changes on the surface. The surface of one of the pressure sensors 16a is attached to a pathway of an entrance or a mouthpiece of the inhalation device, where a user performs a sipping or inhaling action with his/her mouth. Therefore, the inhalation device directly registers a pressure drop when the user is sipping the device. The other pressure sensor 16b is located in another area of the inhalation device which is isolated from the first pressure sensor 16a, and where the pressure is equal with an ambient pressure around the inhalation device. Both pressure sensors 16a, 16b are communicating on the same SPI bus with the controller 18. The controller 18 is continuously asking for pressure information from both pressure sensors 16a, 16b and subtracts the pressure values received from the two pressure sensors 16a, 16b and compares it with pressure difference limits in order to detect a user inhalation.

The invention is, of course, not limited to the preferred embodiments described in detail above, but further variants, modifications and developments are possible within the scope of protection determined by the claims. Furthermore, all embodiments that can be defined by any arbitrary dependent claim combination belong to the invention.

### LIST OF REFERENCE SIGNS

10a, 10b piezoelectric actuator
12a, 12b inductor
14a, 14b switch
16a, 16b pressure sensor
18 controller
20a, 20b output stage
22 shunt resistor
23 shunt resistor
24 amplifier
25 amplifier
26 boost converter
28 linear regulator
30 battery
32 battery charger module
34 connector
36 optical terminal
38 button
50 reference value
52 PI compensator
54 timer module
56 block of the output stage
58 shunt resistance gain
60 shunt amplifier gain
62 integrator
70 idle state
72 overcurrent test
74 piezo regulation
76 overcurrent error
100 piezoelectric actuator
102 reservoir
104 liquid transferring member
105 analogue circuit
106 printed circuit board
107 voltage supply net
108 external memory device
110 housing
120 pod

1. An inhalation device for delivering an atomized mixture of liquids with a regulated mixing ratio, comprising
two reservoirs (102), each storing a respective liquid,
for each reservoir (102) a respective piezoelectric actuator (10a, 10b, 100) for atomizing the respective liquid, and
a pressure sensor (16a, 16b) for detecting a user inhalation,
characterized in that
the inhalation device further comprises a regulating electric circuit for regulating a flow rate of the atomized liquids by controlling the piezoelectric actuators (10a, 10b, 100), wherein the regulating electric circuit comprises a controller (18) and wherein the pressure sensor (16a) is connected to the controller (18) for enabling atomizing only during the user inhalation,
the regulating electric circuit further comprises an output stage (20a, 20b) for each of the piezoelectric actuators (10a, 10b, 100), wherein each output stage (20a, 20b) comprises
an inductor (12a, 12b) forming a resonant circuit with the respective piezoelectric actuator (10a, 10b, 100) having an inherent capacitance, and
a switch (14a, 14b) for driving the output stage (20a, 20b) by a respective output PWM signal having a duty cycle, wherein the output PWM signal is generated by the controller (18),
the mixing ratio and the flow rates of the atomized liquids are determined by the duty cycles of the output PWM signals, and
for each reservoir (102) the inhalation device comprises a respective liquid transferring member (104) made of a capillary material and being in contact with the respective liquid in the reservoir (102) and with the respective piezoelectric actuator (10a, 10b, 100).

2. The inhalation device according to claim 1, characterized in that the regulating electric circuit further comprises a feedback circuit connected to the controller (18) for regulating a state variable characterizing the flow rates of the atomized liquids based on a predetermined reference value of the state variable.

3. The inhalation device according to claim 2, characterized in that the state variable is a current, a voltage, and/or a frequency of the piezoelectric actuator (10a, 10b, 100).

4. The inhalation device according to claim 2, characterized in that the predetermined reference value is chosen from reference values of the state variable that are stored in a look-up table in a memory of the controller (18).

5. The inhalation device according to claim 2, characterized in that the predetermined reference value corresponds to circumstances affecting the flow rate of the atomized liquids, wherein the circumstances include at least one of the following: self-heating of the inhalation device, variations of environmental temperature, moisture, aging of the inhalation device, viscosity of the respective liquid, a material of the respective liquid transferring member (104), contact between the respective liquid transferring member (104) and the respective piezoelectric actuator (10a, 10b, 100), mechanical forces acting up on the inhalation device.

6. The inhalation device according to claim 2, characterized in that the feedback circuit is connected to all of the output stages (20a, 20b) to return feedback from the output stages (20a, 20b) to the controller (18).

7. The inhalation device according to claim 2, characterized in that the regulating electric circuit further comprises a respective feedback circuit for each respective output stage (20a, 20b), and the feedback circuit is connected to the respective output stage (20a, 20b) to return feedback from the respective output stage (20a, 20b) to the controller (18).

8. The inhalation device according to claim 1, characterized in that the piezoelectric actuator (10a, 10b, 100) is a piezoelectric mesh actuator having a first side and an opposite second side and through holes extending between the first side and the second side in a direction of a vibration of the piezoelectric actuator (10a, 10b, 100), wherein ends of the through holes on the first side are in contact with the liquid transferring member (104), and the atomized liquid is generated during vibrations at ends of the through holes on the second side.

9. The inhalation device according to claim 1, characterized by comprising a further pressure sensor (16b) for measuring ambient pressure, wherein user inhalation is determined based on a pressure difference between the pressure sensors (16a, 16b).

10. The inhalation device according to claim 1, characterized in that the switch (14a, 14b) is a MOSFET or a bipolar transistor.

11. The inhalation device according to claim 10, characterized in that the switch (14a, 14b) has a drain-source or a collector-emitter breakdown voltage greater than 200 V.

12. The inhalation device according to claim 1, characterized by further comprising a battery (30), preferably a chargeable battery, connected to the controller (18) for supplying power to the controller (18).

13. The inhalation device according to claim 1, characterized in that the reservoirs (102) are exchangeable and/or refillable.

14. The inhalation device according to claim 1, characterized in that one of the reservoirs (102), the respective piezoelectric actuator (100) is comprised in a pod (120), wherein the pod (120) is exchangeable.

15. The inhalation device according to claim 14, characterized in that the pod (120) further comprises an external memory device (108).
